(19) United States
(12) Patent Application Publication
(10) Pub. No.: US 2019/0038188 A1
(43) Pub. Date: Feb. 7, 2019

(54) Method and apparatus for measuring the Acoustic Reflex with artifact management by using multiple probe tones

(71) Applicant: Andre Lodwig, Woerthsee (DE)

(72) Inventor: Andre Lodwig, Woerthsee (DE)

(73) Assignee: PATH medical GmbH, Germering (DE)

(21) Appl. No.: 15/665,533

(22) Filed: Aug. 1, 2017

(51) Int. Cl. A61B 5/12; A61B 5/00

(52) U.S. Cl. CPC A61B 5/126; A61B 5/6817; A61B 2562/0204; A61B 5/742; A61B 5/7214

(57) ABSTRACT

A method and apparatus is disclosed that performs a measurement of the acoustic reflex. In contrast to prior art, more than one probe tone is used simultaneously to detect the acoustic reflex and separate the reflex response from artifacts. In a preferred configuration, at least one probe tone frequency is selected to be higher than the middle ear resonance frequency.

### CROSS-REFERENCE TO RELATED APPLICATIONS

U.S. Pat. No. 3,395,697 A (1968) “Acoustic reflexometer” discloses a contralateral reflex testing apparatus that detects the acoustic reflex directly as an air pressure variation in the occluded, not stimulated ear canal, without any probe tone.

U.S. Pat. No. 3,757,769 A (1973) “Acoustic admittance testing apparatus” discloses an analog admittance test apparatus which can also detect the acoustic reflex. It is based on a single probe tone of a not specified frequency and implements quadrature detection.

U.S. Pat. No. 3,949,735 A (1976) “Method and apparatus for an ipsilateral reflex test” discloses an apparatus for testing the ipsilateral acoustic reflex. It mixes both a 220 Hz probe tone and 200 ms pulses of a stimulus to one probe speaker. The stimulus timing gates a comparator circuit which compares the filtered microphone signal between stimulated and non-stimulated phase. This appears to be the first ipsilateral acoustic reflex testing apparatus.

U.S. Pat. No. 4,201,225 A (1980) “Method and apparatus for measuring stimulated acoustic reflex latency time” discloses a method to measure the latency of the acoustic reflex with a 220 Hz probe tone

U.S. Pat. No. 4,966,160 A (1990) “Acoustic admittance measuring apparatus with wide dynamic range and logarithmic output”discloses an apparatus for testing the ipsilateral acoustic reflex with a settable, single probe tone in the range of 150 Hz to 2.5 kHz and an exponential detection circuit to allow a wide dynamic range. The inventor states that existing instruments only allow probe tones of 226 and 678 Hz exclusively.

U.S. Pat. No. 6,295,467 B1 (2001) “Method and device for detecting a reflex of the human stapedius muscle” discloses a method to use the stimulus signal itself to detect the ipsilateral reflex, instead of applying a separate probe tone. Pairs of signal bursts are presented within a short period of time, and the recorded sound of both is compared to detect the acoustic reflex, assuming only the latter of the stimuli is impacted by the reflex, due to its latency.

U.S. Pat. No. 8,419,655 B2 (2013) “Method and apparatus for aural acoustic immittance measurement” discloses an apparatus for immittance testing that uses FFT to convert the microphone signal to frequency domain. It still uses a monofrequent probe tone.

US 20130303941 A1 (2013) “Method and Apparatus for Evaluating Dynamic Middle Ear Muscle Activity”discloses a method to test the contralateral acoustic reflex with a broad band signal as the probe tone. Because of the nature of the probe signal, ipsilateral testing is not possible. The method aims at getting more information about the dynamics of the middle ear.

### NON-PATENT REFERENCES

Freeney M., Keefe D. H. (1999). Acoustic Reflex Detection Using Wide-Band Acoustic Reflectance, Admittance, and Power Measurements. Journal of Speech, Language, and Hearing Research Vol. 42, 1029-1041

Peake W T, Rosowski J J. (1997). Acoustic properties of the middle ear. In: M J Crocker (Eds.) Encyclopedia of Acoustics. (pp. 1337-1346) New York: Wiley.

Rosowski J., Wilber L. A. (2015) Acoustic Immittance, Absorbance, and Reflectance in the Human Ear Canal. Semin Hear 2015; 36(01): 11-28

Liu Y-W, Sanford C A, Ellison J C, Fitzpatrick D F, Gorga M P, Keefe D H. (2008) Wideband absorbance tympanometry using pressure sweeps: System development and results on adults with normal hearing. The Journal of the Acoustical Society of America. 2008; 124(6):3708-3719. doi:10.1121/1.3001712.

Wikipedia article on the Acoustic reflex: https://en.wikipedia.org/wiki/Acoustic_reflex

### STATEMENT REGARDING FEDERALLY SPONSORED RESEARCH OR DEVELOPMENT

No federally sponsored research or development is involved in the invention.

### BACKGROUND OF THE INVENTION

The disclosed invention belongs to the field of audiometry. Audiometry, as a branch of audiology, is the science of measuring hearing. A subsection of Audiometry deals with objective methods, which do not need the subject under test to cooperate. One of these methods uses the so-called acoustic reflex for objective measurements.

The acoustic reflex is a middle ear muscle activity, triggered by an acoustic stimulus, which results in a tension to either the ear drum or the ossicles. The effect of this muscle activity is to reduce the mobility of the ear drum. This reduced mobility can, among other methods, be detected acoustically. Sine no cooperative subject feedback is needed, this method therefore belongs to the class of objective hearing tests. A good overall explanation can be found on Wikipedia:

https://en.wikipedia.org/wiki/Acoustic_reflex

A probe for testing the acoustic reflex contains at least one speaker and one microphone. Often, means for applying a static air pressure are also provided. This can be used to compensate middle ear internal pressure in case of tube dysfunction to optimize the general mobility of the ear drum. Therefore, the acoustic reflex test is often included into tympanometers which measure the ear drum mobility as a function of static air pressure. These instruments contain a pump to apply static air pressure to the outer ear. Tympanometry measures the ear drum mobility as a function of static air pressure (relative to ambient pressure), with the best mobility normally occurring at zero relative pressure.

The ear drum mobility is usually described by its “compliance”. Since the ear probe roughly emits a constant volume flow q, independent from the acoustic load, the sound pressure that is measured by the microphone acts inverse to the compliance. A higher compliance, indicating higher mobility, would reduce the recorded probe tone sound level. The instrument is typically calibrated to display the compliance as an equivalent acoustic volume in cm³.

The procedure to detect the acoustic reflex involves the presentation of a low level probe tone, typically at 226 Hz, that is recorded back by the probe's microphone. The reflex is then triggered by an additional acoustic stimulus, either presented at the same ear (ipsilateral) or the opposite ear (contralateral). For ipsilateral reflex tests, a second speaker is often build into the probe to decouple the stimulus from the ongoing probe tone. The probe tone amplitude and/or phase component in the recorded signal changes when the ear drum is stiffened by the acoustic reflex. For a low frequency probe tone, such as 226 Hz, the amplitude of the recorded pilot tone signal would usually rise, indicating a lower compliance. For recording, the microphone signal is usually fed through a narrow band filter that is tuned to the probe tone frequency, to remove the acoustic stimulus (if ipsi-lateral) and noise. This filtering can be implemented analog or digitally. The narrow band filter makes the method relatively robust against external noise. The filter must be wide enough to follow admittance changes fast enough, and narrow enough to provide sufficient noise filtering.

However, certain artifacts, such as movement or swallowing, create changes in the recorded probe tone which can be very similar to those caused by the acoustic reflex. These artifacts basically change the residual volume of the ear canal which results in a probe tone change of the same order as a reflex. The system cannot separate between reflex and artifact; it can only assume there is no reflex when no stimulus was presented. Therefore, suppression or detection of this type of artifacts in reflex testing is desirable to make the test more robust and reliable. This is where the present disclosure steps in.

### BRIEF SUMMARY OF THE INVENTION

The normal ear drum has its resonance at about 1 kHz. This means, at 1 kHz its mobility is highest. During the acoustic reflex, the middle ear gets stiffened, which results in the resonance to move upwards in frequency. FIG. 1 illustrates the general effect. Obviously, the usual probe tone signal frequency of 226 Hz is clearly below the resonance, and the ear drum would appear stiffer when the reflex is active. A probe frequency not too far above the resonance, however, can show an inverted behavior, since the middle ear resonance moves closer to this probe frequency during the reflex.

Real measured data which supports FIG. 1 can be found in Peake W T, Rosowski J J. (1997), pp 1343.

The most typical artifact, caused by mechanical movement, basically modulates the residual ear canal volume (the volume from probe tip to the ear drum). The total compliance will change with this volume change, but in contrast to the reflex, the change will be more similar for various probe frequencies. The effect is illustrated in FIG. 2.

The invention as disclosed here makes use of this effect by applying at least two different probe tones simultaneously to test the reflex. In a preferred configuration, at least one tone would be placed below the normal middle ear resonance frequency, and one above. A reflex would basically cause a decrease of compliance at the lower probe frequency, and an increase at the higher probe frequency. Mechanical artifacts, in contrast to this, would alter both compliance values to the same direction (both up or both down). Therefore, reflex and artifacts can be distinguished from each other, allowing artifact detection and/or suppression.

The principle can both be applied to visual interpretation of the reflex and automated detection. In a visual interpretation setup, curves from different probe tones can be plotted separately, allowing the operator to discriminate the reflex from artifacts. Additionally or alternatively, sum and/or difference traces can be provided where difference traces would contain more reflex activity while sum traces would contain more artifacts.

In an automated setup, the system can evaluate all traces and, by fixed or adaptive rules, judge between reflex and artifacts. An adaptive solution would use no-stimulus periods in the measurement session to evaluate the correlation of different probe tone feedback signals. No-Stimulus periods would just contain artifacts, so that the system can “learn” how artifacts impact the admittances.

The method can as well be applied to record acoustic reflexes from external stimuli, such as cochlea implants.

### BRIEF DESCRIPTION OF THE SEVERAL VIEWS OF THE DRAWING

FIG. 1: illustrates the admittance change with the acoustic reflex: The resonance frequency, which corresponds to a maximum in admittance, moves to higher frequencies. The change in admittance that can be recorded at 3 different frequencies differs. Real, measured data can be found in Peake W T, Rosowski J J. (1997), pp 1343, FIG. 5a.

FIG. 2 illustrates the admittance change with probe movement (typical artifact). The residual volume is modulated, resulting in a similar change in admittance for most frequencies.

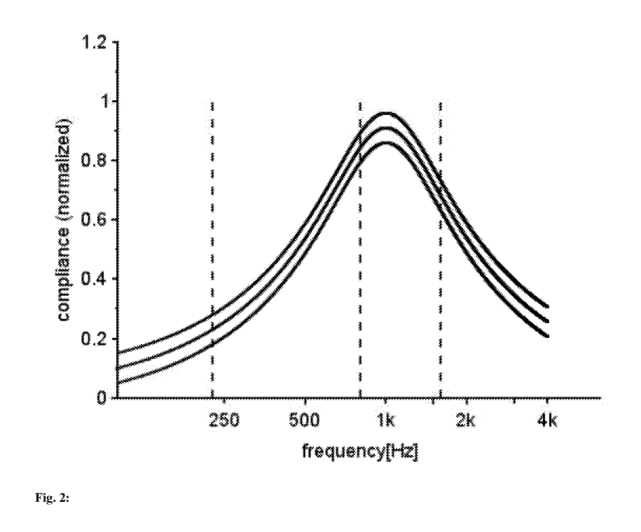

FIG. 3 illustrates a 3-channel recording of a reflex. Since the three traces correspond to different probe frequencies, one of which is above the middle ear resonance frequency, they respond differently to the reflex.

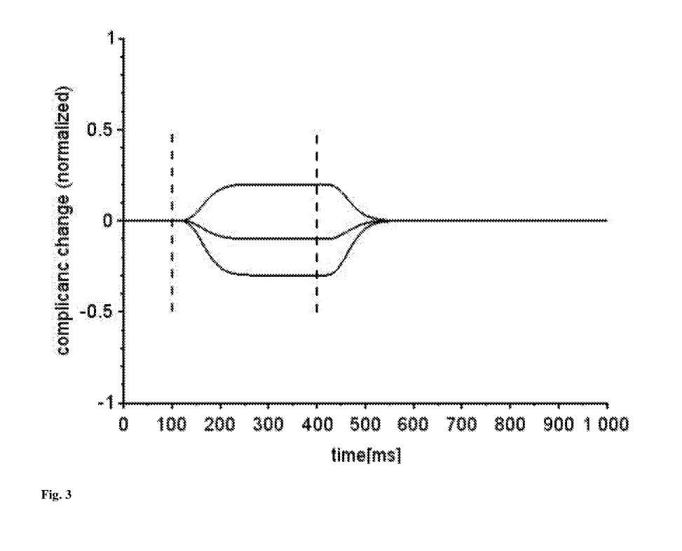

FIG. 4 illustrates a 3-channel recording of volume-changing artifacts. Since a change of the residual volume changes the measured compliance of all frequencies to the same direction, the response of all three channels is to the same sign. Both directions (volume smaller, volume bigger) can occur.

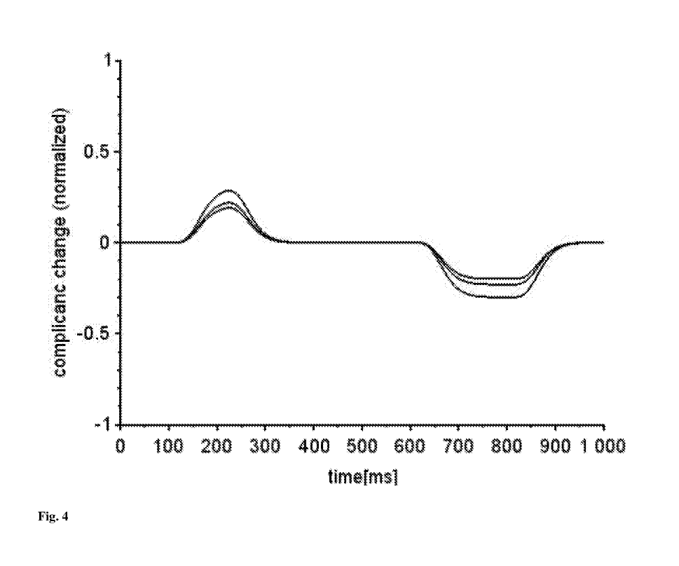

FIG. 5 illustrates a simplified principle schematic of the method. 3 different probe frequencies are generated (1), amplified (2) and presented to the ear probe's (3) speaker (4). The ear probe's microphone (5) records back the signal which is amplified and separated by narrow band filters (6) which are tuned to the probe frequencies. The amplitude of the component frequencies is measured (7), offset removed and presented (8). means to trigger the reflex is not shown for simplicity. It can be acoustically on either ear or electrical (e.g. via a cochlea implant).

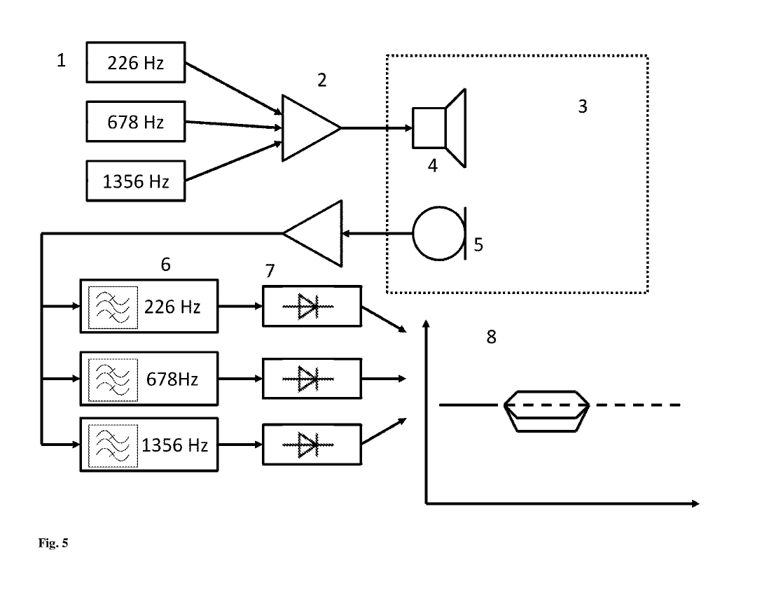

FIG. 6 illustrates a more realistic implementation of the invention, where all signal processing is implemented digitally. A processor (11) is connected to a D/A converter (12) and a A/D converter (16) which provide probe tone and stimulus output and recording of the microphone signal. The processor can be connected to various interfaces (17) which can be a direct user interface, such as display, keyboard, touch-screen, or a connection to a computer for operation. Again, means to trigger the reflex is not shown.

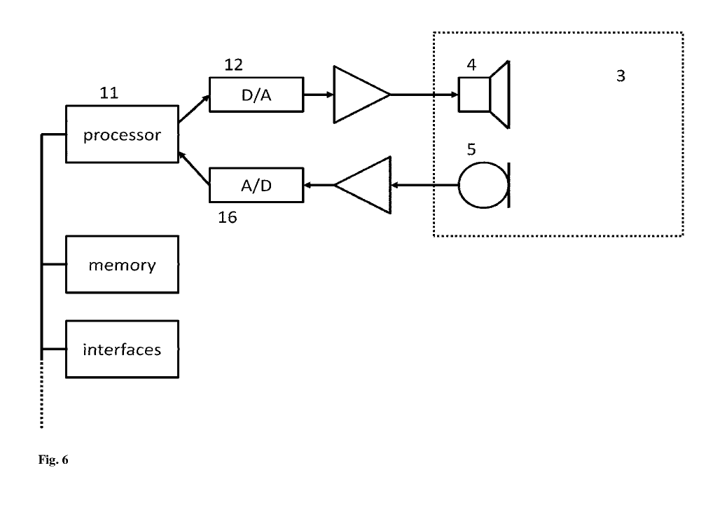

### DETAILED DESCRIPTION OF THE INVENTION

The (human) middle ear transforms the external sound field that enters the ear canal to a sound in the inner ear. Since the inner ear is filled with a fluid, its acoustic impedance is much different, basically towards higher sound pressure and lower medium velocity. The middle ear handles this by connecting a relatively big ear drum membrane to a small stapes footplate, acting as a piston into the inner ear. The middle ear efficiency peaks at a frequency of around 1 kHz. This frequency corresponds to the main resonance of the middle ear, where mobility is highest. The mobility is usually described as compliance, which is in fact the imaginary part of the so-called acoustic admittance. Acoustic impedance is defined as p/q, acoustic admittance as q/p, where p is the sound pressure and q is the sound volume flow (volume/time).

A muscle, called musculus stapedius, is connected to the stapes bone and, if activated, drags the stapes bone sidewards. This temporarily reduces the mobility of the stapes bone and in turn the ear drum. However, since the muscle stiffens the overall middle ear mechanics, it also moves its resonance frequency up.

The musculus stapedius is activated when loud sound is received by either ear, and is thought to be a protective mechanism. Other purposes are under discussion. Since the muscle acts non-voluntary, its activation is called stapedius reflex or acoustic reflex. The sound level that is needed to excite the stapedius reflex is called acoustic reflex threshold.

In audiometry, the acoustic reflex can be used for objective measurements, since the subject under test does not have to voluntary respond to the examiner. This makes this test possible in sleeping subjects, young children, or during surgery. It can also be used in context with implantable hearing devices, such as cochlea implants, which stimulate the inner ear electrically.

The standard method to record the stapedius reflex is to apply a moderate level, low frequency tone of typically 226 Hz to the ear canal via a probe. The probe tone level needs to be well below the reflex threshold. The probe is inserted into the ear canal. The probe also contains a microphone that records back the 226 Hz signal. A stimulus is then applied to the same ear (“ipsilateral”) or the opposite ear (“contralateral”). A separate speaker may be provided for this. The 226 Hz component in the microphone signal will change slightly in level and/or phase while the stapedius reflex is present and temporarily stiffens the ear drum.

Usually, the instruments that record the acoustic reflex are combined in so-called tympanometers. Tympanometry measures the mobility of the ear drum as a function of external static air pressure, and is a diagnostic method for the middle ear. The method to detect the change in mobility via a probe tone is closely related to acoustic reflex test, which is why the methods are often combined. If the tympanometry pump is available in the reflex system, applying a static pressure to a value where the ear drum mobility is maximal, can improve reflex testing reliability. Therefore, before reflex testing, the tympanometry is often executed to determine this pressure of maximum compliance.

In tympanometry, alternate frequencies are often provided which can be used instead of the standard 226 Hz, such as 1 kHz. An alternate approach (“wide band tympanometry”, Liu et. al 2008) uses broad band clicks to calculate the admittance over a broad frequency band as a function of static pressure. A similar approach is proposed by Freeney M., Keefe D. H. (1999). However, the repetition rate of the click is somewhat limited, its sound level needs to be quite high and some averaging is needed to achieve a sufficient signal-to-noise-ratio (SNR). This means the detection signal is at risk to evoke the reflex by itself.

The standard frequency of 226 Hz in tympanometry and reflex testing is historically selected because of a relation between acoustic admittance and effective acoustic volume: At 226 Hz, the acoustic volume in cm³ and the acoustic compliance are numerically equivalent. The frequency of 226 Hz is below the middle ear resonance. The middle ear system acts as a mass-spring-system. At frequencies below the resonance, it mainly acts as a spring, and the reflex will stiffen this spring.

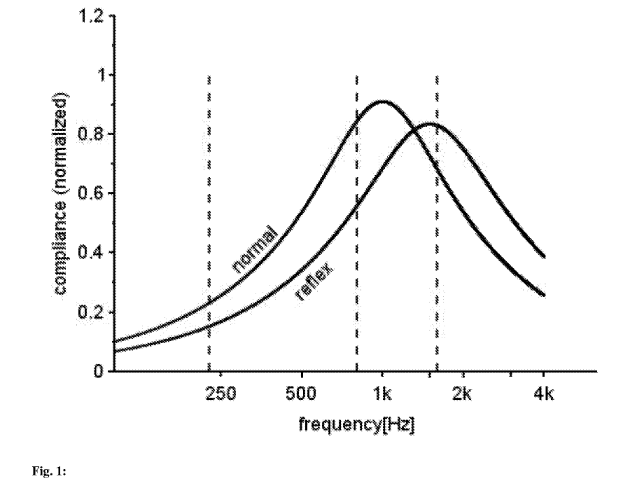

Since the stapedius reflex will shift the middle ear resonance towards higher frequencies, the compliance at a higher probe frequency, where the middle ear mainly acts as a mass, can also rise during the reflex. Therefore, using a probe tone above the middle ear resonance, results in an inverted behavior of the compliance change during the reflex. This is illustrated in FIG. 1.

Since the change in the microphone signal due to the acoustic reflex is small, the measurement can easily be disturbed by artifacts. One obvious artifact is external noise that can interfere with the probe tone recording. This can be handled quite well by using a narrow band filter for the microphone signal. In state-of-the-art implementations, this filtering is implemented digitally. This narrow-band recording allows very moderate probe tone levels, in contrast to wide band approaches.

Another type of artifact includes any movement of the probe position within the ear canal. Such a movement will change the residual volume between probe and ear drum. Since the residual volume contributes to the overall compliance that the probe measures, a change can easily be mistaken as a reflex. A similar artifact occurs if the subject under test swallows or yawns.

As a consequence, an experienced examiner (who is aware of the artifacts) may tend to increase stimulus levels well above the threshold until a clear reflex is recorded. The threshold would then be overestimated. It may also be necessary to repeat testing specific stimuli until artifact-free recordings are achieved, thus prolonging total examination time.

An artifact as described above will modulate the residual volume. The compliance change during the artifact can therefore be expected to be similar for various probe frequencies. In contrast to this, the acoustic reflex, since it shifts the middle ear resonance, will act differently for different probe frequencies.

The idea of the present invention is to use this different behavior for artifact suppression. It presents more than one probe tone frequency simultaneously, and records all of them back. The different probe frequencies can be separated via dedicated narrow band filters from the common microphone signal, so that more than one compliance values can be derived simultaneously at any time. The overall loudness of the tones can still be kept moderate enough to be safe against triggering the reflex.

If, for example, frequencies 226, 678, 1356 Hz (1, 3, 7 times 226 Hz) are used, compliance at 226 would drop, compliance at 678 would not change much, and compliance at 1356 would rise during the acoustic reflex. In contrast to this, the measured compliance at all frequencies would rise if the probe is moved outwards or drop if it is moved inwards the ear canal, due to a mere residual volume change.

The test system can therefore separate artifacts and reflex in the response data. This separation can either be done by the examiner, who observes, for example, 3 traces instead of just one, knowing how reflex response and artifact response look like. The data can also be evaluated automatically, and a derived, artifact-free reflex signal shown to the examiner. Such evaluation may contain calculation of differences of compliance traces, correlation methods, etc. It may also make use of the phases of the recorded signals. A preferred implementation weights the recorded traces with factors (some of which are negative), which are selected to result in an artifact-free reflex response trace.

An apparatus which implements the method would typically contain a processor and digital-analog and analog-digital conversion. All signal generation and recording is implemented digitially. The filters would typically be implemented using quadrature detection, which allows phase-locked recording. In such an implementation, the number of probe tones that are used simultaneously is only limited by the overall sound level they produce, which needs to be below the reflex threshold, and the processing power of the digital system. A typical implementation would use 3 probe tones.

The configuration of the probe tones (number and frequencies) can be configurable, to, for example, optimally handle middle ears of different age groups or middle ear disorders. Said factors for deriving an artifact-free signal could also be adjustable or even automatically “learned” by the system during no-stimulus phases.

1. A method and apparatus to measure the acoustic reflex that presents and records at least two probe tones of different frequencies simultaneously, comprising
a. a probe with at least one speaker and at least one microphone, to be placed in the human ear canal
b. means for signal generation of said probe tones and presentation via said loudspeaker(s)
c. means for recording back said probe tones via said microphone(s)
d. evaluation of said recorded probe tones for amplitudes and/or phases to detect reflex and artifacts

2. A method and apparatus according to claim 1 which provides means for presentation of a stimulus, either ipsilateral or contralateral or both, to trigger the acoustic reflex

3. A method and apparatus according to claim 1, that provides an interface for an external trigger to allow detection of the acoustic reflex from external stimulation

4. A method and apparatus according to claim 1, where signal generation and analysis are implemented digitally

5. A method and apparatus according to claim 1, which displays the compliance as derived from the recorded probe tones or other features of those signals graphically for visual inspection

6. A method and apparatus according to claim 1, which automatically detects the acoustic reflex by using two or more of the recorded probe tone signals simultaneously

7. A method and apparatus according to claim 1, which automatically detects or suppresses artifacts by using two or more of the recorded probe tone signals

8. A method and apparatus according to claim 1, which automatically adapts artifact suppression parameters during no-stimulus phases

9. A method and apparatus according to claim 1, that also evaluates the phase of the recorded tones to separate reflex from artifact response

10. A method and apparatus according to claim 1, that includes a tympanometric pump to allow reflex testing under static pressure

11. A method and apparatus according to claim 6, that automatically selects stimulus frequencies and/or levels to automatically find the subject's reflex threshold
